(19) United States
(12) Patent Application Publication
(10) Pub. No.: US 2026/0335668 A1
(43) Pub. Date: Oct. 29, 2026

(54) Phototherapy eye mask

(71) Applicant: Dongguan Laiguang Electronic Technology Co., ltd., Dongguan (CN)

(72) Inventor: Zekai Luo, Dongguan (CN)

(21) Appl. No.: 19/281,521

(22) Filed: Jul. 25, 2025

(30) Foreign Application Priority Data
Apr. 28, 2025 (CN) 2025208292355

(51) Int. Cl. A61N 5/06; A61M 21/00; A61M 21/02

(52) U.S. Cl. CPC A61N 5/0613; A61M 21/02; A61M 2021/0055; A61N 2005/0648; A61N 2005/0654; A61N 2005/0663

(57) ABSTRACT

The present application provides a phototherapy eye mask, which includes an arc-shaped eye mask, a light-transmitting member, a lamp body and a wearing member. The light-transmitting member is provided on the side of the arc-shaped eye mask facing the user and is connected to the arc-shaped eye mask. The lamp body is located between the light-transmitting member and the arc-shaped eye mask. The lamp body is provided opposite to the light-transmitting member, and the light-emitting end of the lamp body faces the light-transmitting member. The light output from the light-emitting end of the lamp body passes through the light-transmitting member and irradiates the skin around the eyes of the user. The wearing member is connected to the arc-shaped eye mask and worn on the user's head. The wearing member can be adapted to different sizes of heads.

### CROSS-REFERENCE TO RELATED APPLICATIONS

The present application claims priority to a Chinese patent application No. 202520829235.5, titled “Phototherapy Eye Mask”, filed on Apr. 28, 2025, the entire contents of which are incorporated herein by reference.

### TECHNICAL FIELD

The present application relates to the technical field of phototherapy eye masks, in particular to a phototherapy eye mask.

### BACKGROUND

With the development of technology, phototherapy eye masks have been applied in people's daily lives and are worn on people's faces. Phototherapy eye masks are used to perform phototherapy on the skin around the eyes of the user by utilizing phototherapy light.

In the related art, the existing phototherapy eye mask includes an arc-shaped eye mask, a light-transmitting member and a lamp body. The light-transmitting member is connected to the arc-shaped eye mask. The lamp body is located between the light-transmitting member and the arc-shaped eye mask, and is provided opposite to the light-transmitting member. A light-emitting end of the lamp body faces the light-transmitting member. The light output from the light-emitting end of the lamp body passes through the light-transmitting member and irradiates the skin around the eyes of the user. However, during the use process, the phototherapy eye mask is fixed on the user's head by the user's hand supporting it, which leads to the poor convenience of use of the existing phototherapy eye mask.

### SUMMARY

The purpose of the present application is to provide a phototherapy eye mask. The light-transmitting member is provided on the side of the arc-shaped eye mask facing the user and is connected to the arc-shaped eye mask. The lamp body is located between the light-transmitting member and the arc-shaped eye mask. The lamp body is provided opposite to the light-transmitting member, and a light-emitting end of the lamp body faces the light-transmitting member. The light output from the light-emitting end of the lamp body passes through the light-transmitting member and irradiates the skin around the eyes of the user. The wearing member is connected to the arc-shaped eye mask and is worn on the user's head. The wearing member can be adapted to different sizes of heads. By means of the wearing member, the phototherapy eye mask can be adapted to different sizes of heads, so that the phototherapy eye mask can be fixed on different sizes of heads, which avoids the need for the user to support the phototherapy eye mask with their hands to fix it on the head, thus improving the convenience of using the phototherapy eye mask.

To achieve the above purpose, the present application provides the following technical solution: a phototherapy eye mask is used for performing phototherapy on the skin around the eyes of the user. The phototherapy eye mask includes:
an arc-shaped eye mask;
a light-transmitting member, provided on a side of the arc-shaped eye mask facing the user and connected to the arc-shaped eye mask; and
a lamp body, located between the light-transmitting member and the arc-shaped eye mask,
where the lamp body is provided opposite to the light-transmitting member, and a light-emitting end of the lamp body faces the light-transmitting member, and light output from the light-emitting end of the lamp body passes through the light-transmitting member and irradiates the skin around the eyes of the user; and
a wearing member, connected to the arc-shaped eye mask and worn on the user's head and adapted to different sizes of heads

The present application provides a phototherapy eye mask. The light-transmitting member is provided on the side of the arc-shaped eye mask facing the user and is connected to the arc-shaped eye mask. The lamp body is located between the light-transmitting member and the arc-shaped eye mask. The lamp body is provided opposite to the light-transmitting member, and the light-emitting end of the lamp body faces the light-transmitting member. The light output from the light-emitting end of the lamp body passes through the light-transmitting member and irradiates the skin around the eyes of the user. The wearing member is connected to the arc-shaped eye mask and worn on the user's head. The wearing member can be adapted to different sizes of heads. The phototherapy eye mask can be made to fit different sizes of heads through the wearing member, making it convenient to fix the phototherapy eye mask on heads of different sizes, which avoids the need to fix the phototherapy eye mask on the user's head by the user's hand supporting it, thus improving the convenience of using the phototherapy eye mask.

### BRIEF DESCRIPTION OF THE DRAWINGS

In order to more clearly illustrate the technical solutions in the embodiments of the present application, the following will briefly introduce the drawings that need to be used in the description of the embodiments. Obviously, the drawings in the following description are only some embodiments of the present application. For those skilled in the art, without creative efforts, other drawings can also be obtained based on these drawings.

In order to understand the present application and its beneficial effects more completely, the following will be described in conjunction with the drawings. The same reference numerals in the following description represent the same parts.

FIG. 1 shows a schematic diagram of the wearing member of the phototherapy eye mask being a knob-type strap in the embodiment of the present application.

FIG. 2 shows a schematic diagram of the wearing member of the phototherapy eye mask being an elastic strap in the embodiment of the present application.

FIG. 3 shows a cross-sectional view of the phototherapy eye mask in the embodiment of the present application.

FIG. 4 shows an exploded view of the phototherapy eye mask in the embodiment of the present application.

FIG. 5 shows a partial enlarged view of part A in FIG. 4.

FIG. 6 shows a schematic diagram of a partial structure of the phototherapy eye mask in the embodiment of the present application.

FIG. 7 shows a schematic diagram of the connection of the lamp body and the conductive member of the phototherapy eye mask in the embodiment of the present application.

FIG. 8 shows a schematic diagram of the connection of the lamp body and the conductive member of the phototherapy eye mask in the embodiment of the present application.

### DETAILED DESCRIPTION OF THE EMBODIMENTS

The following will clearly and completely describe the technical solutions in the embodiments of the present application with reference to the accompanying drawings in the embodiments of the present application. Obviously, the described embodiments are only a part of the embodiments of the present application, not all of them. Based on the embodiments in the present application, all other embodiments obtained by those skilled in the art without creative efforts shall fall within the protection scope of the present application.

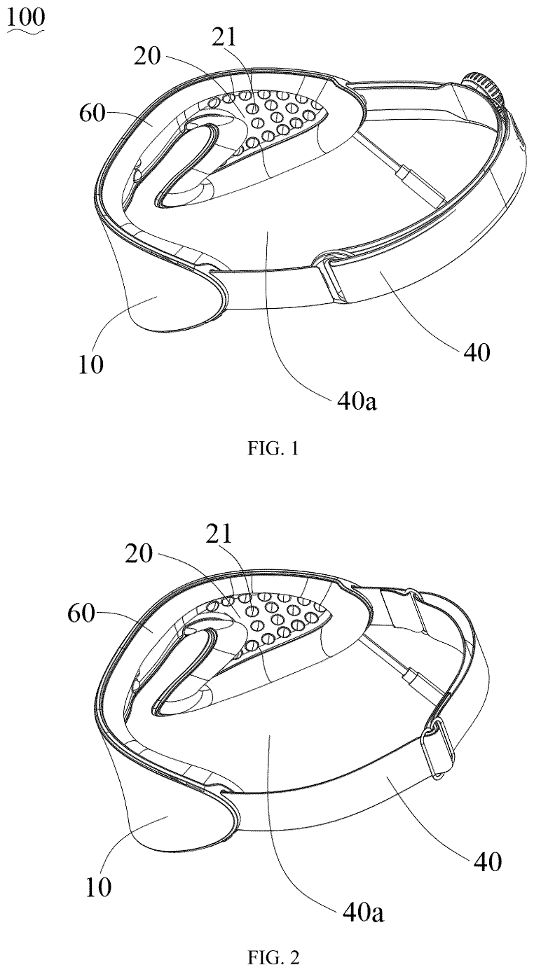

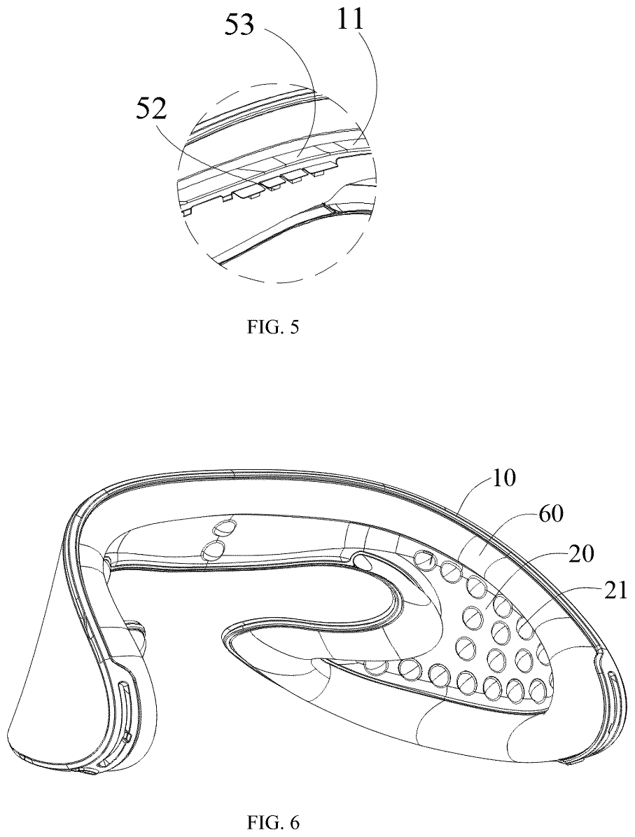

Please refer to FIGS. 1-7, the embodiment of the present application provides a phototherapy eye mask 100, which is used to perform phototherapy on the skin around the eyes of the user with phototherapy light.

Please refer to FIGS. 1-7, in the embodiment of the present application, the phototherapy eye mask 100 includes an arc-shaped eye mask 10, a light-transmitting member 20, a lamp body 30, and a wearing member 40. The light-transmitting member 20 is provided on a side of the arc-shaped eye mask 10 facing the user and is connected to the arc-shaped eye mask 10. The lamp body 30 is located between the light-transmitting member 20 and the arc-shaped eye mask 10. The lamp body 30 is provided opposite to the light-transmitting member 20, and the light-emitting end of the lamp body 30 faces the light-transmitting member 20. The light output from the light-emitting end of the lamp body 30 passes through the light-transmitting member 20 and irradiates the skin around the eyes of the user. The wearing member 40 is connected to the arc-shaped eye mask 10 and is worn on the user's head. The wearing member 40 can be adapted to different sizes of heads. The phototherapy eye mask 100 can be adapted to different sizes of heads through the wearing member 40, making it convenient to fix the phototherapy eye mask 100 on heads of different sizes, which avoids the need to fix the phototherapy eye mask 100 on the user's head by the user's hand supporting it, thus improving the convenience of using the phototherapy eye mask 100.

Please refer to FIGS. 1-7, in the embodiment of the present application, the arc-shaped eye mask 10 serves as a supporting component of the phototherapy eye mask 100. The arc-shaped eye mask 10 is used to support the light-transmitting member 20, the lamp body 30, and the wearing member 40. The arc-shaped eye mask 10 is provided relative to the eyes of the user, which facilitates the arc-shaped eye mask 10 to be fixed on the eyes or head of the user.

The light-transmitting member 20 is provided on the side of the arc-shaped eye mask 10 facing the user and is connected to the arc-shaped eye mask 10, so that the light-transmitting member 20 can be fixed on the side of the arc-shaped eye mask 10 facing the user.

The lamp body 30 is located between the light-transmitting member 20 and the arc-shaped eye mask 10. The lamp body 30 is provided opposite to the light-transmitting member 20, and the light-emitting end of the lamp body 30 faces the light-transmitting member 20. The light output from the light-emitting end of the lamp body 30 passes through the light-transmitting member 20 and irradiates the skin around the eyes of the user, which facilitates the light output from the light-emitting end of the lamp body 30 to be transmitted to the skin around the eyes of the user, thus facilitating the use of the light output from the light-emitting end of the lamp body 30 to perform phototherapy on the skin around the eyes of the user.

The wearing member 40 is provided on the rear side of the arc-shaped eye mask 10. The wearing member 40 is connected to the arc-shaped eye mask 10 and is worn on the user's head. The wearing member 40 can be adapted to different sizes of heads. The phototherapy eye mask 100 can be adapted to different sizes of heads through the wearing member 40, making it convenient to fix the phototherapy eye mask 100 on heads of different sizes, which avoids the need to fix the phototherapy eye mask 100 on the user's head by the user's hand supporting it, thus improving the convenience of using the phototherapy eye mask 100.

Please refer to FIGS. 1-4. in the embodiment of the present application, the wearing member 40 is provided on the rear side of the arc-shaped eye mask 10, and a wearing space 40a is formed between the wearing member 40 and the arc-shaped eye mask 10. The wearing space 40a is adapted to different sizes of heads. By adjusting the length of the wearing member 40, the size of the wearing space 40a can be adjusted, so that the wearing space 40a can accommodate different sizes of heads, which facilitates the arc-shaped eye mask 10 and the wearing member 40 to be closely attached to the user's head, so as to fix the phototherapy eye mask 100 on the user's head. Optionally, the wearing member 40 is an elastic strap, a knob-type strap, or an adjustable strap, which facilitates the length adjustment of the wearing member 40.

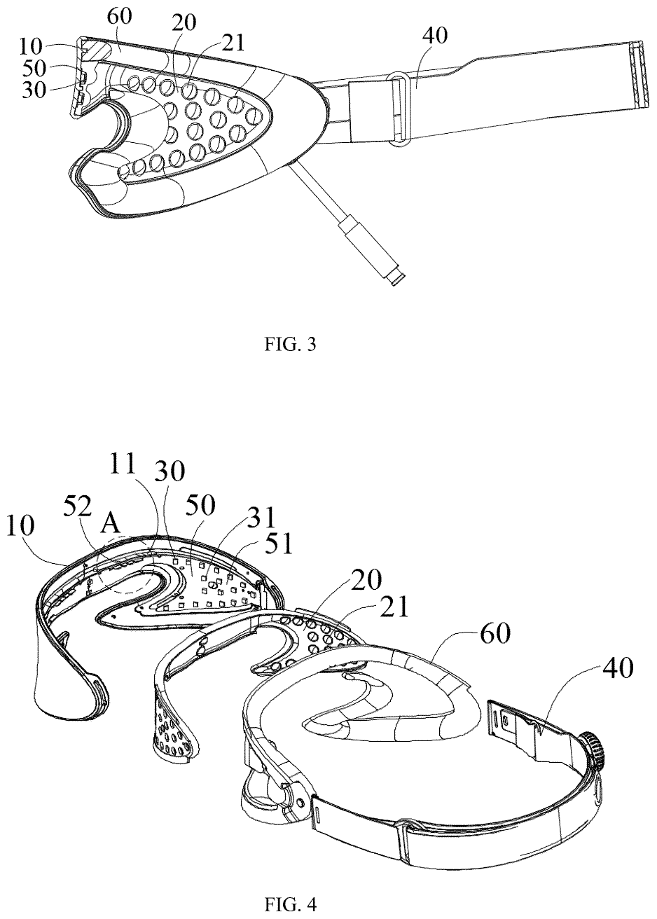

Please refer to FIGS. 3-6, in the embodiment of the present application, the lamp body 30 is at least one of a phototherapy lamp bead, a white-light lamp bead, and a red-light lamp bead. When the lamp body 30 is a phototherapy lamp bead, it facilitates the lamp body 30 to perform phototherapy on the user. When the lamp body 30 is a white-light lamp bead, it facilitates the lamp body 30 to refresh the user. When the lamp body 30 is a red-light lamp bead, it facilitates the lamp body 30 to help the user sleep.

Please refer to FIGS. 3-6, in the embodiment of the present application, the phototherapy eye mask 100 further includes a conductive member 50. The conductive member 50 is located between the light-transmitting member 20 and the arc-shaped eye mask 10. There are a plurality of lamp bodies 30. All of the plurality of lamp bodies 30 are electrically connected to the conductive member 50 and are located at different positions of the conductive member 50, which facilitates the conductive member 50 to support the plurality of lamp bodies 30 at the same time. The light-emitting ends of the plurality of lamp bodies 30 output lights of different colors towards the light-transmitting main body and perform a phototherapy effect on the skin around the eyes of the user, which facilitates the plurality of lamp bodies 30 to output lights of different colors to different positions of the skin around the eyes of the user, so as to achieve the plurality of phototherapy effects.

Optionally, the conductive member 50 is a flexible circuit board. The flexible circuit board has a deformation effect, which facilitates the flexible circuit board to adapt to the arc-shaped contour of the light-transmitting member 20 or the arc-shaped contour of the arc-shaped eye mask 10. The flexible circuit board is connected to the light-transmitting member 20 or the arc-shaped eye mask 10, which facilitates the flexible circuit board to be fixed on the light-transmitting member 20 or the arc-shaped eye mask 10. A power-supply end of the lamp body 30 is electrically connected to the flexible circuit board, which facilitates the lamp body 30 to be started to output light through the flexible circuit board. A light-emitting end of the lamp body 30 faces the lens 21 and is wrapped by the lens 21, which facilitates the light output from the light-emitting end of the lamp body 30 to be diffused or concentrated by the lens 21, avoiding the direct irradiation of the light output from the lamp body 30 to the skin around the eyes of the user, improving the irradiation effect of the light-transmitting component 100. And the lens 21 has a protective effect on the lamp body 30, thereby improving the phototherapy effect of the lamp body 30 on the user's skin.

When the flexible circuit board is connected to the arc-shaped eye mask 10, the flexible circuit board is provided with the plurality of positioning holes. The side wall of the arc-shaped eye mask 10 facing the light-transmitting member 20 is protruded with the plurality of positioning columns. Each positioning column passes through the corresponding positioning hole and is blocked by the light-transmitting member 20, which facilitates the flexible circuit to be positioned and connected to the arc-shaped eye mask 10 through the cooperation of the the plurality of positioning holes and the plurality of positioning columns, ensuring the position accuracy of the flexible circuit relative to the arc-shaped eye mask 10 and avoiding the position offset of the flexible circuit relative to the arc-shaped eye mask 10.

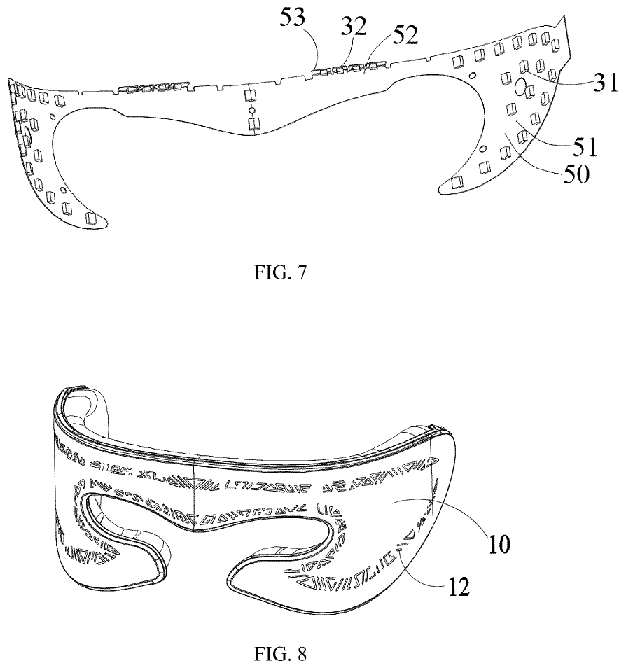

Please refer to FIG. 7, in the embodiment of the present application, the plurality of lamp bodies 30 include a first lamp body 31. The conductive member 50 is provided with a first phototherapy region 51. The first lamp body 31 is distributed in the first phototherapy region 51. The first phototherapy region 51 is provided relative to the user's canthus. The first lamp body 31 is a phototherapy lamp bead. The first lamp body 31 is used to output phototherapy light to the skin around the eyes of the user, which facilitates the first lamp body 31 to be electrically connected to the conductive member 50 through the first phototherapy region 51, so that the first lamp body 31 can be provided around the eyes of the user, and then the first lamp body 31 can output light towards the skin around the eyes of the user, so as to realize the light therapy of the first lamp body 31 on the skin around the eyes of the user.

Please refer to FIG. 7, in the embodiment of the present application, the plurality of lamp bodies 30 include a second lamp body 32. The conductive member 50 is provided with a second phototherapy region 52. The second phototherapy region 52 is staggered with the first phototherapy region 51. The second phototherapy region 52 is provided relative to the user's eyebrows. The second lamp body 32 is distributed in the second phototherapy region 52, which facilitates the second lamp body 32 and the first lamp body 31 to be in different positions. The second lamp body 32 is provided with a red-light emitting unit or a white-light emitting unit, so that the second phototherapy region 52 is a refreshing region or a sleep-aiding region.

When the second lamp body 32 is provided with a red-light emitting unit, it facilitates the second lamp body 32 to promote blood circulation in the skin around the eyes of the user, so as to improve the sleep quality. When the second lamp body 32 is provided with a white-light emitting unit, it facilitates the second lamp body 32 to relieve anxiety in the eyes of the user, so as to achieve the refreshing effect. Optionally, when the second lamp body 32 is provided with a white-light emitting unit, the light output from the white-light emitting unit irradiates the eyes of the user.

Referring to FIG. 8, in addition, the arc-shaped eye mask 10 is provided with a light-transmitting surface 12. The light-transmitting surface 12 is a part of the arc-shaped eye mask 10 and allows the light output from the second lamp body 32 or the first lamp body 31 to be presented outward. Optionally, the light-transmitting surface 12 can be designed with patterns, so that when the light is presented relative to the light-transmitting surface 12, the gorgeous effect and the appearance effect of the arc-shaped eye mask 10 are improved. Optionally, the light-transmitting surface 12 is provided along the outer contour of the arc-shaped eye mask 10.

Optionally, the conductive member 50 is provided with a conductive arm 53. The side wall of the arc-shaped eye mask 10 facing the light-transmitting member 20 is protruded with a boss 11. The boss 11 supports the conductive arm 53, which facilitates the conductive arm 53 to be fixed on the arc-shaped eye mask 10 through the boss 11, ensuring the position accuracy of the conductive arm 53 relative to the arc-shaped eye mask 10.

In the embodiment of the present application, there are plurality of conductive arms 53. The plurality of conductive arms 53 are provided along the boss 11. Each second lamp body 32 is installed on the corresponding conductive arm 53. By arranging plurality of conductive arms 53, the plurality of second lamp bodies 32 can be connected.

Please refer to FIGS. 4-6, in the embodiment of the present application, the light-transmitting member 20 is provided with a lens 21. The lens 21 is protruded from the surface of the light-transmitting member 20. The lamp body 30 is provided opposite to the lens 21. The light-emitting end of the lamp body 30 faces the lens 21. The light output from the light-emitting end of the lamp body 30 is conducted outward by the lens 21. The lens 21 is integrated with the light-transmitting member 20, which facilitates the light output from the light-emitting end of the lamp body 30 to be diffused or concentrated by the lens 21, avoiding the direct irradiation of the light output from the lamp body 30 to the eyes of the user, improving the irradiation effect of the phototherapy eye mask 100. And the lens 21 has a protective effect on the lamp body 30, thereby improving the phototherapy effect of the lamp body 30 on the user's skin.

Please refer to FIGS. 4-6, in the embodiment of the present application, the light-transmitting member 20 is a light-transmitting silicone member. The lens 21 is separately connected to the light-transmitting member 20, or the lens 21 is integrally connected to the light-transmitting member 20. When the lens 21 is separately connected to the light-transmitting member 20, it facilitates the lens 21 to be connected to or detached from the light-transmitting member 20, thereby improving the convenience of replacing the lens 21 and reducing the maintenance cost of the light-transmitting member 20. When the lens 21 is integrally connected to the light-transmitting member 20, the connection stability between the lens 21 and the light-transmitting member 20 is ensured, the connection strength between the lens 21 and the light-transmitting member 20 is improved, and the detachment of the lens 21 and the light-transmitting member 20 during use is avoided.

Meanwhile, in the embodiment of the present application, the lens 21 is of an arc-shaped, spherical, or special-shaped manner, which facilitates the inner contour of the lens 21 to adapt to the arc-shaped lamp body 30, the spherical lamp body 30, or the special-shaped lamp body 30, improving the versatility of the lens 21. Optionally, the lens 21 is a condensing lens 21, which facilitates the light output from the light-emitting end of the lamp body 30 to be concentrated by the lens 21. The lens 21 can cover the corresponding lamp body 30.

Please refer to FIGS. 1-5, in the embodiment of the present application, the phototherapy eye mask 100 further includes a flexible pad 60. The flexible pad 60 is connected to the arc-shaped eye mask 10 and can be flexibly attached to the user's skin. The flexible pad 60 is provided along the outer contour of the arc-shaped eye mask 10. The light-transmitting member 20 is located on one side of the arc-shaped eye mask 10, which facilitates the arc-shaped eye mask 10 to be in contact with the user through the flexible pad 60, so that the arc-shaped eye mask 10 can be in flexible contact with the user, avoiding the hard contact between the arc-shaped eye mask 10 and the user, and improving the user's comfort when using the phototherapy eye mask 100.

The flexible pad 60 is provided along the outer contour of the arc-shaped eye mask 10. A cross-section of the flexible pad 60 is arc-shaped and is of a continuous ring shape, and can be in elastic contact with the user's skin. The flexible pad 60 is attached to the user's skin and constructs a sealed space along the outer contour of the arc-shaped eye mask 10, preventing foreign objects from entering the space enclosed by the flexible pad 60 from the external environment and protecting the light-transmitting member 20 in this space.

Compared with the related art, the beneficial effects of the present application are as follows:

The present application provides a phototherapy eye mask 100. The light-transmitting member 20 is provided on the side of the arc-shaped eye mask 10 facing the user and is connected to the arc-shaped eye mask 10. The lamp body 30 is located between the light-transmitting member 20 and the arc-shaped eye mask 10. The lamp body 30 is provided opposite to the light-transmitting member 20, and the light-emitting end of the lamp body 30 faces the light-transmitting member 20. The light output from the light-emitting end of the lamp body 30 passes through the light-transmitting member 20 and irradiates the skin around the eyes of the user. The wearing member 40 is connected to the arc-shaped eye mask 10 and is worn on the user's head. The wearing member 40 can be adapted to different sizes of heads. The phototherapy eye mask 100 can be adapted to different sizes of heads through the wearing member 40, making it convenient to fix the phototherapy eye mask 100 on heads of different sizes which avoids the need to fix the phototherapy eye mask 100 on the user's head by the user's hand supporting it, thus improving the convenience of using the phototherapy eye mask 100.

It should be noted that all directional indications (such as up, down, left, right, front, back . . . ) in the embodiments of the present application are only used to explain the relative positional relationship and movement conditions of various components in a specific posture. If this specific posture changes, the directional indications will also change accordingly.

It should also be noted that when an element is referred to as being “fixed on” or “provided on” another element, it can be directly on the other element or there may be an intermediate element at the same time. When an element is referred to as being “connected” to another element, it can be directly connected to the other element or indirectly connected to the other element through an intermediate element.

In addition, the descriptions involving “first”, “second”, etc. in the present application are only for the purpose of description and cannot be understood as indicating or implying their relative importance or implicitly indicating the number of the indicated technical features. Therefore, the features defined with “first” and “second” may explicitly or implicitly include at least one of these features. In addition, the technical solutions between various embodiments can be combined with each other, but it must be based on what can be achieved by those skilled in the art. When the combination of technical solutions is contradictory or cannot be achieved, it should be considered that such a combination of technical solutions does not exist and is not within the scope of protection required by the present application.

The above is only the preferred embodiment of the present application, and it does not limit the patent scope of the present application. Under the inventive concept of the present application, any equivalent structural transformation made by using the contents of the description and the accompanying drawings of the present application, or any direct/indirect application in other related technical fields, is included in the patent protection scope of the present application.

What is claimed is:

1. A phototherapy eye mask, for phototherapy of a skin around eyes of a user, comprising:
an arc-shaped eye mask;
a light-transmitting member, provided on a side of the arc-shaped eye mask facing the user and connected to the arc-shaped eye mask; and
a lamp body, located between the light-transmitting member and the arc-shaped eye mask,
wherein the lamp body is provided opposite to the light-transmitting member, and a light-emitting end of the lamp body faces the light-transmitting member, and light output from the light-emitting end of the lamp body passes through the light-transmitting member and irradiates the skin around the eyes of the user; and
a wearing member, connected to the arc-shaped eye mask and worn on the user's head and adapted to different sizes of heads.

2. The phototherapy eye mask according to claim 1, wherein the wearing member is provided at a rear side of the arc-shaped eye mask, and a wearing space is formed between the wearing member and the arc-shaped eye mask, and the wearing space is adapted to different sizes of heads.

3. The phototherapy eye mask according to claim 2, wherein the wearing member is an elastic strap, a knob-type strap or an adjustable strap.

4. The phototherapy eye mask according to claim 1, wherein the lamp body is at least one of a phototherapy lamp bead, a white light lamp bead and a red light lamp bead.

5. The phototherapy eye mask according to claim 1, further comprising a conductive member,
wherein the conductive member is located between the light-transmitting member and the arc-shaped eye mask,
a plurality of lamp bodies are provided, all of the plurality of lamp bodies are electrically connected to the conductive member and are located at different positions of the conductive member, and the light-emitting ends of the plurality of lamp bodies output lights of different colors towards the light-transmitting member and perform a phototherapy effect on the skin around the eyes of the user.

6. The phototherapy eye mask according to claim 5, wherein the plurality of lamp bodies comprise a first lamp body;
the conductive member is provided with a first phototherapy region, and the first lamp body is distributed in the first phototherapy region; and
the first lamp body is a phototherapy lamp bead, and the first lamp body is configured for outputting phototherapy light to the skin around the eyes of the user.

7. The phototherapy eye mask according to claim 5, wherein the plurality of lamp bodies comprise a second lamp body;
the conductive member is provided with a second phototherapy region, and the second lamp body is distributed in the second phototherapy region; and
the second lamp body is provided with a red light emitting unit or a white light emitting unit, so that the second phototherapy region is a refreshing region or a sleep-aiding region.

8. The phototherapy eye mask according to claim 1, wherein the light-transmitting member is provided with a lens, and the lens is protruded from a surface of the light-transmitting member;
the lamp body is provided opposite to the lens, and the light-emitting end of the lamp body faces the lens, and the light output from the light-emitting end of the lamp body is conducted outwards by the lens.

9. The phototherapy eye mask according to claim 8, wherein the light-transmitting member is a light-transmitting silicone member;
the lens is of an arc shape or a spherical shape and covers the corresponding lamp body;
the lens is separately connected to the light-transmitting member, or the lens is integrally connected to the light-transmitting member.

10. The phototherapy eye mask according to claim 1, further comprising a flexible pad,
wherein the flexible pad is connected to the arc-shaped eye mask and flexibly fit the user's skin;
the flexible pad is provided along an outer contour of the arc-shaped eye mask, and the light-transmitting member is located on one side of the arc-shaped eye mask; and
the flexible pad fits the user's skin and constructs a sealed space along the outer contour of the arc-shaped eye mask to prevent foreign objects from entering the sealed space enclosed by the flexible pad from an external environment and protect the light-transmitting member located in the sealed space.

11. The phototherapy eye mask according to claim 5, wherein the conductive member is provided with a conductive arm, and a boss is protruded from a side wall of the arc-shaped eye mask facing the light-transmitting member, and the boss supports the conductive arm.

12. A phototherapy eye mask, for phototherapy of a skin around eyes of a user, comprising:
an arc-shaped eye mask;
a light-transmitting member, provided on a side of the arc-shaped eye mask facing the user and connected to the arc-shaped eye mask; and
a lamp body, located between the light-transmitting member and the arc-shaped eye mask;
wherein the lamp body is provided opposite to the light-transmitting member, and a light-emitting end of the lamp body faces the light-transmitting member, and light output from the light-emitting end of the lamp body passes through the light-transmitting member and irradiates the skin around the eyes of the user.

13. The phototherapy eye mask according to claim 12, further comprising a conductive member, wherein the conductive member is located between the light-transmitting member and the arc-shaped eye mask, a plurality of lamp bodies are provided, all of the plurality of lamp bodies are electrically connected to the conductive member and are located at different positions of the conductive member, and the light-emitting ends of the plurality of lamp bodies output lights of different colors towards the light-transmitting member and perform a phototherapy effect on the skin around the eyes of the user.

14. The phototherapy eye mask according to claim 13, wherein the plurality of lamp bodies comprise a first lamp body; the conductive member is provided with a first phototherapy region, and the first lamp body is distributed in the first phototherapy region; the first lamp body is a phototherapy lamp bead, and the first lamp body is configured for outputting phototherapy light to the skin around the eyes of the user.

15. The phototherapy eye mask according to claim 13, wherein the plurality of lamp bodies comprise a second lamp body;
the conductive member is provided with a second phototherapy region, and the second lamp body is distributed in the second phototherapy region; and
the second lamp body is provided with a red light emitting unit or a white light emitting unit, so that the second phototherapy region is a refreshing region or a sleep-aiding region.

16. The phototherapy eye mask according to claim 12, wherein the light-transmitting member is provided with a lens, and the lens is protruded from a surface of the light-transmitting member; and
the lamp body is provided opposite to the lens, and the light-emitting end of the lamp body faces the lens, and the light output from the light-emitting end of the lamp body is conducted outwards by the lens.

17. The phototherapy eye mask according to claim 16, wherein the light-transmitting member is a light-transmitting silicone member;
the lens is of an arc shape or a spherical shape and covers the corresponding lamp body; and
the lens is separately connected to the light-transmitting member, or the lens is integrally connected to the light-transmitting member.

18. A phototherapy eye mask, for phototherapy of a skin around eyes of a user, comprising:
an arc-shaped eye mask;
a light-transmitting member, provided on a side of the arc-shaped eye mask facing the user and connected to the arc-shaped eye mask; and
a lamp body, located between the light-transmitting member and the arc-shaped eye mask;
wherein the lamp body is provided opposite to the light-transmitting member, and a light-emitting end of the lamp body faces the light-transmitting member;
light output from the light-emitting end of the lamp body passes through the light-transmitting member and irradiates the skin around the eyes of the user;
wherein the phototherapy eye mask further comprises a conductive member, the conductive member is located between the light-transmitting member and the arc-shaped eye mask, the conductive member is provided with a conductive arm, and a boss is protruded from a side wall of the arc-shaped eye mask facing the light-transmitting member, and the boss supports the conductive arm.

19. The phototherapy eye mask according to claim 18, wherein a plurality of lamp bodies comprise a first lamp body;
the conductive member is provided with a first phototherapy region, and the first lamp body is distributed in the first phototherapy region; and
the first lamp body is a phototherapy lamp bead, and the first lamp body is configured for outputting phototherapy light to the skin around the eyes of the user.

20. The phototherapy eye mask according to claim 18, wherein the plurality of lamp bodies comprise a second lamp body;
the conductive member is provided with a second phototherapy region, and the second lamp body is distributed in the second phototherapy region; and
the second lamp body is provided with a red light emitting unit or a white light emitting unit, so that the second phototherapy region is a refreshing region or a sleep-aiding region.
